(19) United States
(12) Patent Application Publication
(10) Pub. No.: US 2008/0194772 A1
(43) Pub. Date: Aug. 14, 2008

(54) Method For Producing an Aqueous Polymer Dispersion

(75) Inventors: Xiang-Ming Kong, Mainz (DE); Motonori Yamamoto, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/910,567

(22) PCT Filed: Apr. 7, 2006
(86) PCT No.: PCT/EP2006/061418

§ 371 Date: Oct. 3, 2007

(30) Foreign Application Priority Data
Apr. 7, 2005 (DE) 102005016226.6

(51) Int. Cl. C08G 69/02; C12P 13/02; C08G 69/04

(52) U.S. Cl. 525/426; 435/129; 525/420

(57) ABSTRACT

A process for preparing an aqueous polymer dispersion, in which, in an aqueous medium, in a first reaction stage, a diamine compound and a dicarboxylic acid compound are reacted in the presence of an enzyme and of a dispersant, and also, if appropriate, of a low water solubility organic solvent and/or of an ethylenically unsaturated monomer, to give a polyamide, and thereafter, in the presence of the polyamide, in a second reaction stage, an ethylenically unsaturated monomer is free-radically polymerized.

The present invention provides a process for preparing an aqueous polymer dispersion, which comprises reacting, in an aqueous medium, in a first reaction stage,
a) a diamine compound A and
b) a dicarboxylic acid compound B,
   in the presence
c) of an enzyme C which catalyzes a polycondensation reaction of diamine compound A and dicarboxylic acid compound B, and
d) of a dispersant D,
   and also, if appropriate,
e) of a low water solubility organic solvent E and/or
f) of an ethylenically unsaturated monomer F,
   to give a polyamide, and thereafter, in the presence of the polyamide, in a second reaction stage, free-radically polymerizing an ethylenically unsaturated monomer F.

The present invention also provides for the aqueous polymer dispersions obtainable by the process according to the invention, the polymer powders obtainable therefrom, and for the use thereof.

Processes for preparing aqueous polyamide dispersions are common knowledge. The preparation is generally effected in such a way that an organic diamine and an organic dicarboxylic acid are converted to a polyamide. This polyamide is then generally first converted to a polyamide melt in a subsequent stage and the melt is then dispersed in an aqueous medium to form what is known as a secondary dispersion with the aid of organic solvents and/or dispersants by various methods. When a solvent is used, it has to be distilled off again after the dispersion step (on this subject, see, for example, DE-B 1028328, U.S. Pat. No. 2,951,054, U.S. Pat. No. 3,130,181, U.S. Pat. No. 4,886,844, U.S. Pat. No. 5,236,996, U.S. Pat. No. 6,777,488, WO 97/47686 or WO 98/44062). A patent application filed by this applicant at the German Patent and Trademark Office with the application reference number DE 10 2004 058 072.3 discloses the direct, enzyme-catalyzed preparation of aqueous polyamide dispersions starting from diamine compounds and dicarboxylic acid compounds.

The aqueous polyamide dispersions obtainable by the known processes, and the polyamides themselves, have advantageous properties in many applications, although there is nevertheless frequently further need for optimization.

It was an object of the present invention to provide a process for preparing new types of aqueous polymer dispersions based on polyamide compounds.

Surprisingly, the object was achieved by the process defined at the outset.

Useful diamine compounds A are any organic diamine compounds which have two primary or secondary amino groups, of which preference is given to primary amino groups. The organic basic skeleton having the two amino groups may have a C₂-C₂₀ aliphatic, C₃-C₂₀ cycloaliphatic, aromatic or heteroaromatic structure. Examples of compounds having two primary amino groups are 1,2-diaminoethane, 1,3-diaminopropane, 1,2-diaminopropane, 2-methyl-1,3-diaminopropane, 2,2-dimethyl-1,3-diaminopropane (neopentyldiamine), 1,4-diaminobutane, 1,2-diaminobutane, 1,3-diaminobutane, 1-methyl-1,4-diaminobutane, 2-methyl-1,4-diaminobutane, 2,2-dimethyl-1,4-diaminobutane, 2,3-dimethyl-1,4-diaminobutane, 1,5-diaminopentane, 1,2-diaminopentane, 1,3-diaminopentane, 1,4-diaminopentane, 2-methyl-1,5-diaminopentane, 3-methyl-1,5-diaminopentane, 2,2-dimethyl-1,5-diaminopentane, 2,3-dimethyl-1,5-diaminopentane, 2,4-dimethyl-1,5-diaminopentane, 1,6-diaminohexane, 1,2-diaminohexane, 1,3-diaminohexane, 1,4-diaminohexane, 1,5-diaminohexane, 2-methyl-1,5-diaminohexane, 3-methyl-1,5-diaminohexane, 2,2-dimethyl-1,5-diaminohexane, 2,3-dimethyl-1,5-diaminohexane, 3,3-dimethyl-1,5-diaminohexane, N,N′-dimethyl-1,6-diaminohexane, 1,7-diaminoheptane 1,8-diaminooctane, 1,9-diaminononane, 1,10-diaminodecane, 1,11-diaminoundecane, 1,12-diaminododecane, 1,2-diaminocyclohexane, 1,3-diaminocyclohexane, 1,4-diaminocyclohexane, 3,3′-diaminodicyclohexylmethane, 4,4′-diaminodicyclohexylmethane (dicyan), 3,3′-dimethyl-4,4′-diaminodicyclohexylmethane (Laromin®), isophoronediamine (3-aminomethyl-3,5,5-trimethylcyclohexylamine), 1,4-diazine (piperazine), 1,2-diaminobenzene, 1,3-diaminobenzene, 1,4-diaminobenzene, m-xylylenediamine [1,3-(diaminomethyl)benzene] and p-xylylenediamine [1,4-(diaminomethyl)benzene]. It will be appreciated that it is also possible to use mixtures of the above compounds.

Preference is given to using 1,6-diaminohexane, 1,12-diaminododecane, 2,2-dimethyl-1,3-diaminopropane, 1,4-diaminocyclohexane, isophoronediamine, 3,3′-diaminodicyclohexylmethane, 4,4′-diaminodicyclohexylmethane, 3,3′-dimethyl-4,4′-diaminodicyclohexylmethane, m-xylylenediamine and/or p-xylylenediamine.

The dicarboxylic acid compounds B used may in principle be any C₂-C₄₀ aliphatic, C₃-C₂₀ cycloaliphatic, aromatic or heteroaromatic compounds which have two carboxylic acid groups (carboxyl groups) or derivatives thereof. The derivatives which find use are in particular C₁-C₁₀-alkyl, preferably methyl, ethyl, n-propyl or isopropyl, mono- or diesters of the aforementioned dicarboxylic acids, the corresponding dicarbonyl halides, in particular the dicarbonyl chlorides and the corresponding dicarboxylic anhydrides. Examples of such compounds are ethanedioic acid (oxalic acid), propanedioic acid (malonic acid), butanedioic acid (succinic acid), pentanedioic acid (glutaric acid), hexanedioic acid (adipic acid), heptanedioic acid (pimelic acid), octanedioic acid (suberic acid), nonanedioic acid (azelaic acid), decanedioic acid (sebacic acid), undecanedioic acid, dodecanedioic acid, tridecanedioic acid (brassylic acid), C₃₂ dimer fatty acid (commercial product from Cognis Corp., USA) benzene-1,2-dicarboxylic acid (phthalic acid), benzene-1,3-dicarboxylic acid (isophthalic acid) or benzene-1,4-dicarboxylic acid (terephthalic acid), the methyl esters thereof, for example dimethyl ethanedioate, dimethyl propanedioate, dimethyl butanedioate, dimethyl pentanedioate, dimethyl hexanedioate, dimethyl heptanedioate, dimethyl octanedioate, dimethyl nonanedioate, dimethyl decanedioate, dimethyl undecanedioate, dimethyl dodecanedioate, dimethyl tridecanedioate, C₃₂-dimer fatty acid dimethyl ester, dimethyl phthalate, dimethyl isophthalate or dimethyl terephthalate, the dichlorides thereof, for example ethanedioyl chloride, propanedioyl chloride, butanedioyl chloride, pentanedioyl chloride, hexanedioyl chloride, heptanedioyl chloride, octanedioyl chloride, nonanedioyl chloride, decanedioyl chloride, undecanedioyl chloride, dodecanedioyl chloride, tridecanedioyl chloride, C₃₂-dimer fatty acid chloride, phthaloyl chloride, isophthaloyl chloride or terephthaloyl chloride, and the anhydrides thereof, for example butanedicarboxylic anhydride, pentanedicarboxylic anhydride or phthalic anhydride. It will be appreciated that it is also possible to use mixtures of the above compounds B.

Preference is given to using the dicarboxylic acids, especially butanedioic acid, hexanedioic acid, decanedioic acid, dodecanedioic acid, terephthalic acid and/or isophthalic acid or the corresponding dimethyl esters thereof.

According to the invention, the quantitative ratios of diamine compound A and of the dicarboxylic acid compound B are selected in such a way that the molar ratio of dicarboxylic acid compound B to diamine compound A is from 0.5 to 1.5, generally from 0.8 to 1.3, frequently from 0.9 to 1.1 and often from 0.95 to 1.05. It is particularly favorable when the molar ratio is 1, i.e. just as many amino groups are present as carboxyl groups or groups derived therefrom (for example ester groups [—CO₂-alkyl] or carbonyl halides [—CO-Hal]).

It is essential to the process that the reaction of diamine compound A with dicarboxylic acid compound B proceeds in aqueous medium in the presence of an enzyme C which catalyzes a polycondensation reaction of diamine compound A and dicarboxylic acid compound B. A polycondensation reaction refers to a reaction of the amino groups from the diamine compound A with the carboxyl groups, or the groups derived therefrom, from the dicarboxylic acid compound B with elimination of water (dicarboxylic acids or dicarboxylic anhydrides), alcohols (esters) or hydrogen halide (carbonyl halides) to form a polyamide.

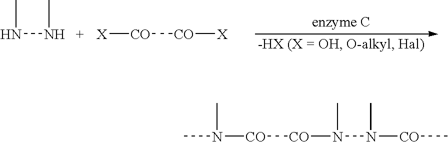

In this reaction, the enzyme C used may in principle be any enzyme which is capable of catalyzing a polycondensation reaction of diamine compound A and dicarboxylic acid compound B in aqueous medium. Especially suitable as enzyme C are hydrolases [EC 3.x.x.x], for example, esterases [EC 3.1.x.x], proteases [EC 3.4.x.x] and/or hydrolases which react with C—N bonds other than peptide bonds. According to the invention, carboxylesterases [EC 3.1.1.1] and/or lipases [EC 3.1.1.3] in particular are used. Examples thereof are lipases from Achromobacter sp., Aspergillus sp., Candida sp., Candida antarctica, Mucor sp., Penicilium sp., Geotricum sp., Rhizopus sp, Burkholderia sp., Pseudomonas sp., Pseudomonas cepacia, Thermomyces sp., porcine pancreas or wheatgerms, and carboxylesterases from Bacillus sp., Pseudomonas sp., Burkholderia sp., Mucor sp., Saccharomyces sp., Rhizopus sp., Thermoanaerobium sp., porcine liver or equine liver. It will be appreciated that it is possible to use a single enzyme C or a mixture of different enzymes C. It is also possible to use the enzymes C in free and/or immobilized form. Preference is given to using lipase from Pseudomonas cepacia, Burkholderia platarii or Candida antarctica in free and/or immobilized form (for example Novozym® 435 from Novozymes A/S, Denmark).

The total amount of enzymes C used is generally from 0.001 to 40% by weight, frequently from 0.1 to 15% by weight and often from 0.5 to 8% by weight, based in each case on the sum of the total amounts of diamine compound A and dicarboxylic acid compound B.

The dispersants D used in the process according to the invention may in principle be emulsifiers and/or protective colloids. It is self-evident that the emulsifiers and/or protective colloids are selected so as to be compatible especially with the enzymes C used and not to deactivate them. Which emulsifiers and/or protective colloids can be used for a certain enzyme C is known to or can be determined by those skilled in the art in simple preliminary experiments.

Suitable protective colloids are, for example, polyvinyl alcohols, polyalkylene glycols, alkali metal salts of polyacrylic acids and polymethacrylic acids, gelatin derivatives or copolymers comprising acrylic acid, methacrylic acid, maleic anhydride, 2-acrylamido-2-methylpropanesulfonic acid and/or 4-styrenesulfonic acid, and alkali metal salts thereof, but also homo- and copolymers comprising N-vinylpyrrolidone, N-vinyl-caprolactam, N-vinylcarbazole, 1-vinylimidazole, 2-vinylimidazole, 2-vinylpyridine, 4-vinylpyridine, acrylamide, methacrylamide, amine-bearing acrylates, methacrylates, acrylamides and/or methacrylamides. A comprehensive description of further suitable protective colloids can be found in Houben-Weyl, Methoden der organischen Chemie [Methods of Organic Chemistry], volume XIV/1, Makromolekulare Stoffe [Macromolecular substances], Georg-Thieme-Verlag, Stuttgart, 1961, p. 411 to 420.

It will be appreciated that mixtures of protective colloids and/or emulsifiers may also be used. Frequently, the dispersants used are exclusively emulsifiers whose relative molecular weights, in contrast to the protective colloids, are typically below 1000. They may be of anionic, cationic or nonionic nature. In the case of the use of mixtures of interface-active substances, it will be appreciated that the individual components have to be compatible with one another, which can be checked in the case of doubt by a few preliminary experiments. In general, anionic emulsifiers are compatible with one another and with nonionic emulsifiers. The same also applies to cationic emulsifiers, while anionic and cationic emulsifiers are usually not compatible with one another. An overview of suitable emulsifiers can be found in Houben-Weyl, Methoden der organischen Chemie, volume XIV/1, Makromolekulare Stoffe [Macromolecular substances], Georg-Thieme-Verlag, Stuttgart, 1961, p. 192 to 208.

According to the invention, the dispersants D used are in particular emulsifiers.

Useful nonionic emulsifiers are, for example, ethoxylated monoalkylphenols, dialkylphenols and trialkylphenols (EO units: 3 to 50, alkyl radical: C₄ to Cl₂) and ethoxylated fatty alcohols (EO units: 3 to 80; alkyl radical: C₈ to C₃₆). Examples of such emulsifiers are the Lutensol® A brands (C₁₂C₁₄ fatty alcohol ethoxylates, EO units: 3 to 8), Lutensol® AO brands (C₁₃C₁₅ oxo alcohol ethoxylates, EO units: 3 to 30), Lutensol® AT brands (C₁₆C₁₈ fatty alcohol ethoxylates, EO units: 11 to 80), Lutensol® ON brands (C₁₀ oxo alcohol ethoxylates, EO units: 3 to 11) and the Lutensol® TO brands (C₁₃ oxo alcohol ethoxylates, EO units: 3 to 20) from BASF AG.

Customary anionic emulsifiers are, for example, alkali metal and ammonium salts of alkyl sulfates (alkyl radical: C₈ to C₁₂), of sulfuric monoesters of ethoxylated alkanols (EO units: 4 to 30, alkyl radical: C₁₂ to C₁₈) and ethoxylated alkylphenols (EO units: 3 to 50, alkyl radical: C₄ to C₁₂), of alkylsulfonic acids (alkyl radical: C₁₂ to C₁₈) and of alkylarylsulfonic acids (alkyl radical: C₉ to C₁₈).

Further anionic emulsifiers which have been found to be useful are compounds of the general formula (I)

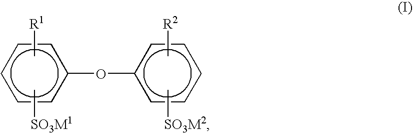

where R¹ and R² are each hydrogen atoms or C₄- to C₂₄-alkyl and are not both hydrogen atoms, and M¹ and M² may be alkali metal ions and/or ammonium ions. In the general formula (I), R¹ and R² are preferably linear or branched alkyl radicals having from 6 to 18 carbon atoms, in particular having 6, 12 or 16 carbon atoms, or hydrogen, but R¹ and R² are not both hydrogen atoms. M¹ and M² are preferably sodium, potassium or ammonium, of which sodium is particularly preferred. Particularly advantageous compounds (I) are those in which M¹ and M² are each sodium, R¹ is a branched alkyl radical having 12 carbon atoms and R² is a hydrogen atom or R¹. Frequently, technical-grade mixtures which have a proportion of from 50 to 90% by weight of the monoalkylated product are used, for example Dowfax® 2A1 (brand of Dow Chemical Company). The compounds (I) are common knowledge, for example from U.S. Pat. No. 4,269,749, and are commercially available.

Suitable cation-active emulsifiers are generally primary, secondary, tertiary or quaternary ammonium salts having a C₆- to C₁₈-alkyl, C₆- to C₁₈-alkylaryl or heterocyclic radical, alkanolammonium salts, pyridinium salts, imidazolinium salts, oxazolinium salts, morpholinium salts, thiazolinium salts and salts of amine oxides, quinolinium salts, isoquinolinium salts, tropylium salts, sulfonium salts and phosphonium salts. Examples include dodecylammonium acetate or the corresponding sulfate, the sulfates or acetates of the various 2-(N,N,N-trimethylammonium)ethyl-paraffinic esters, N-cetylpyridinium sulfate, N-laurylpyridinium sulfate and N-cetyl-N,N,N-trimethylammonium sulfate, N-dodecyl-N,N,N-trimethylammonium sulfate, N-octyl-N,N,N-trimethylammonium sulfate, N,N-distearyl-N,N-dimethylammonium sulfate and also the Gemini surfactant N,N′-(lauryldimethyl)ethylenediamine disulfate, ethoxylated tallow fat alkyl-N-methylammonium sulfate and ethoxylated oleylamine (for example Uniperol® AC from BASF AG, approx. 12 ethylene oxide units). Numerous further examples can be found in H. Stache, Tensid-Taschenbuch [Surfactants Handbook], Carl-Hanser-Verlag, Munich, Vienna, 1981, and in McCutcheon's, Emulsifiers & Detergents, MC Publishing Company, Glen Rock, 1989. It is important that the anionic countergroups have a very low nucleophilicity, for example perchlorate, sulfate, phosphate, nitrate and carboxylates, for example acetate, trifluoroacetate, trichloroacetate, propionate, oxalate, citrate, benzoate, and also conjugate anions of organic sulfonic acids, for example methylsulfonate, trifluoromethylsulfonate and paratoluenesulfonate, and also tetrafluoroborate, tetraphenylborate, tetrakis(pentafluorophenyl)borate, tetrakis[bis(3,5-trifluoromethyl)phenyl]borate, hexafluorophosphate, hexafluoroarsenate or hexafluoroantimonate.

The emulsifiers which are used with preference as dispersants D are advantageously used in a total amount of from 0.005 to 20% by weight, preferably from 0.01 to 15% by weight, in particular from 0.1 to 10% by weight, based in each case on the sum of the total amounts of diamine compound A and dicarboxylic acid compound B.

The total amount of the protective colloids used as dispersants D in addition to or instead of the emulsifiers is often from 0.1 to 10% by weight and frequently from 0.2 to 7% by weight, based in each case on the sum of the total amounts of diamine compound A and dicarboxylic acid compound B.

However, preference is given to using emulsifiers, especially nonionic emulsifiers, as dispersants D.

According to the invention, low water solubility organic solvents E and/or ethylenically unsaturated monomers F may optionally additionally be used in the first reaction stage.

Suitable solvents E are liquid aliphatic and aromatic hydrocarbons having from 5 to 30 carbon atoms, for example n-pentane and isomers, cyclopentane, n-hexane and isomers, cyclohexane, n-heptane and isomers, n-octane and isomers, n-nonane and isomers, n-decane and isomers, n-dodecane and isomers, n-tetradecane and isomers, n-hexadecane and isomers, n-octadecane and isomers, benzene, toluene, ethylbenzene, cumene, o-, m- or p-xylene, mesitylene, and generally hydrocarbon mixtures in the boiling range of from 30 to 250° C. It is likewise possible to use hydroxyl compounds such as saturated and unsaturated fatty alcohols having from 10 to 28 carbon atoms, for example n-dodecanol, n-tetradecanol, n-hexadecanol and isomers thereof, or cetyl alcohol, esters, for example fatty acid esters having from 10 to 28 carbon atoms in the acid moiety and from 1 to 10 carbon atoms in the alcohol moiety, or esters of carboxylic acids and fatty alcohols having from 1 to 10 carbon atoms in the carboxylic acid moiety and from 10 to 28 carbon atoms in the alcohol moiety. It will be appreciated that it is also possible to use mixtures of the aforementioned solvents.

The total amount of solvent is up to 60% by weight, preferably from 0.1 to 40% by weight and especially preferably from 0.5 to 10% by weight, based in each case on the total amount of water in the first reaction stage.

In the context of this document, low water solubility solvent E shall be understood to mean that the solvent E or the mixture of solvent E in deionized water at 20° C. and 1 atm (absolute) has a solubility of ≦50 g/l, preferably ≦10 g/l and advantageously ≦5 g/l.

Useful ethylenically unsaturated monomers F include in principle all free-radically polymerizable ethylenically unsaturated compounds. Useful monomers F include, in particular, easily free-radically polymerizable ethylenically unsaturated monomers, for example ethylene, vinylaromatic monomers such as styrene, α-methylstyrene, o-chlorostyrene or vinyltoluenes, esters of vinyl alcohol and monocarboxylic acids having from 1 to 18 carbon atoms, such as vinyl acetate, vinyl propionate, vinyl n-butyrate, vinyl laurate and vinyl stearate, esters of α,β-monoethylenically unsaturated mono- and dicarboxylic acids preferably having from 3 to 6 carbon atoms, such as, in particular, acrylic acid, methacrylic acid, maleic acid, fumaric acid and itaconic acid, with alkanols having generally from 1 to 12, preferably from 1 to 8 and in particular from 1 to 4, carbon atoms, such as particularly methyl, ethyl, n-butyl, isobutyl and 2-ethylhexyl acrylate and methacrylate, dimethyl maleate and di-n-butyl maleate, nitriles of α,β-monoethylenically unsaturated carboxylic acids, such as acrylonitrile, and C₄₋₈ conjugated dienes such as 1,3-butadiene and isoprene. It will be appreciated that it is also possible to use mixtures of the aforementioned monomers F. These monomers F generally constitute the principal monomers which, based on the total amount of the monomers F to be polymerized by the process according to the invention, normally account for a proportion of ≧50% by weight, preferably ≧80% by weight or advantageously ≧90% by weight. In general, these monomers are only of moderate to low solubility in water under standard conditions [20° C., 1 atm (absolute)].

Further monomers F which typically increase the internal strength of the polymer obtainable by polymerization of the ethylenically unsaturated monomers F normally have at least one epoxy, hydroxyl, N-methylol or carbonyl group, or at least two nonconjugated ethylenically unsaturated double bonds. Examples thereof are monomers having two vinyl radicals, monomers having two vinylidene radicals, and monomers having two alkenyl radicals. Particularly advantageous in this context are the diesters of dihydric alcohols with α,β-monoethylenically unsaturated monocarboxylic acids, among which acrylic and methacrylic acid are preferred. Examples of such monomers having two nonconjugated ethylenically unsaturated double bonds are alkylene glycol diacrylates and dimethacrylates such as ethylene glycol diacrylate, 1,2-propylene glycol diacrylate, 1,3-propylene glycol diacrylate, 1,3-butylene glycol diacrylate, 1,4-butylene glycol diacrylates and ethylene glycol dimethacrylate, 1,2-propylene glycol dimethacrylate, 1,3-propylene glycol dimethacrylate, 1,3-butylene glycol dimethacrylate, 1,4-butylene glycol dimethacrylate, and also divinylbenzene, vinyl methacrylate, vinyl acrylate, allyl methacrylate, allyl acrylate, diallyl maleate, diallyl fumarate, methylenebisacrylamide, cyclopentadienyl acrylate, triallyl cyanurate, and triallyl isocyanurate. Of particular significance in this context are also the C₁-C₈-hydroxyalkyl methacrylates and acrylates, such as n-hydroxyethyl, n-hydroxypropyl or n-hydroxybutyl acrylate and methacrylate, and compounds such as diacetoneacrylamide and acetylacetoxyethyl acrylate and methacrylate. In accordance with the invention, the aforementioned monomers, based on the total amount of the ethylenically unsaturated monomers F, are used in amounts of up to 5% by weight, frequently from 0.1% to 3% by weight, and often from 0.5% to 2% by weight.

The monomers F used can also be ethylenically unsaturated monomers comprising siloxane groups, such as the vinyltrialkoxysilanes, for example vinyltrimethoxysilane, alkylvinyidialkoxysilanes, acryloyloxyalkyltrialkoxysilanes, or methacryloyloxyalkyltrialkoxysilanes, for example acryloyloxyethyltrimethoxysilane, methacryloyloxyethyltrimethoxysilane, acryloyloxypropyltrimethoxysilane or methacryloyloxypropyltrimethoxysilane. These monomers are used in total amounts of up to 5% by weight, frequently from 0.01% to 3% by weight, and often from 0.05% to 1% by weight, based in each case on the total amount of the monomers F.

As well as these, the monomers F used can additionally be those ethylenically unsaturated monomers FS which either comprise at least one acid group and/or its corresponding anion or those ethylenically unsaturated monomers FA which comprise at least one amino, amido, ureido or N-heterocyclic group and/or the N-protonated or N-alkylated ammonium derivatives thereof. Based on the total amount of the monomers F to be polymerized, the amount of monomers FS or monomers FA, respectively, is up to 10% by weight, often from 0.1 to 7% by weight, and frequently from 0.2 to 5% by weight.

The monomers FS used are ethylenically unsaturated monomers having at least one acid group. The acid group may, for example, be a carboxylic, sulfonic, sulfuric, phosphoric and/or phosphonic acid group. Examples of such monomers FS are acrylic acid, methacrylic acid, maleic acid, fumaric acid, itaconic acid, crotonic acid, 4-styrenesulfonic acid, 2-methacryloyloxyethylsulfonic acid, vinylsulfonic acid, and vinylphosphonic acid, and also phosphoric monoesters of n-hydroxyalkyl acrylates and n-hydroxyalkyl methacrylates, for example phosphoric monoesters of hydroxyethyl acrylate, n-hydroxypropyl acrylate, n-hydroxybutyl acrylate and hydroxyethyl methacrylate, n-hydroxypropyl methacrylate or n-hydroxybutyl methacrylate. In accordance with the invention, however, it is also possible to use the ammonium and alkali metal salts of the aforementioned ethylenically unsaturated monomers having at least one acid group. Preferred alkali metals are in particular sodium and potassium. Examples of such compounds are the ammonium, sodium, and potassium salts of acrylic acid, methacrylic acid, maleic acid, fumaric acid, itaconic acid, crotonic acid, 4-styrenesulfonic acid, 2-methacryloyloxyethylsulfonic acid, vinylsulfonic acid, and vinylphosphonic acid, and also the mono- and diammonium, -sodium and -potassium salts of the phosphoric monoesters of hydroxyethyl acrylate, n-hydroxypropyl acrylate, n-hydroxybutyl acrylate and hydroxyethyl methacrylate, n-hydroxypropyl methacrylate or n-hydroxybutyl methacrylate.

Preference is given to using, as monomers FS, acrylic acid, methacrylic acid, maleic acid, fumaric acid, itaconic acid, crotonic acid, 4-styrenesulfonic acid, 2-methacryloyloxyethylsulfonic acid, vinylsulfonic acid, and vinylphosphonic acid.

The monomers FA used are ethylenically unsaturated monomers which comprise at least one amino, amido, ureido or N-heterocyclic group, and/or the N-protonated or N-alkylated ammonium derivatives thereof.

Examples of monomers FA which comprise at least one amino group are 2-aminoethyl acrylate, 2-aminoethyl methacrylate, 3-aminopropyl acrylate, 3-aminopropyl methacrylate, 4-amino-n-butyl acrylate, 4-amino-n-butyl methacrylate, 2-(N-methylamino)ethyl acrylate, 2-(N-methylamino)ethyl methacrylate, 2-(N-ethylamino)ethyl acrylate, 2-(N-ethylamino)ethyl methacrylate, 2-(N-n-propylamino)ethyl acrylate, 2-(N-n-propylamino)ethyl methacrylate, 2-(N-isopropylamino)ethyl acrylate, 2-(N-isopropylamino)ethyl methacrylate, 2-(N-tert-butylamino)ethyl acrylate, 2-(N-tert-butylamino)ethyl methacrylate (available commercially, for example, as Norsocryl® TBAEMA from Elf Atochem), 2-(N,N-dimethylamino)ethyl acrylate (available commercially, for example, as Norsocryl® ADAME from Elf Atochem), 2-(N,N-dimethylamino)ethyl methacrylate (available commercially, for example, as Norsocryl® MADAME from Elf Atochem), 2-(N,N-diethylamino)ethyl acrylate, 2-(N,N-diethylamino)ethyl methacrylate, 2-(N,N-di-n-propylamino)ethyl acrylate, 2-(N,N-di-n-propylamino)ethyl methacrylate, 2-(N,N-diisopropylamino)ethyl acrylate, 2-(N,N-diisopropylamino)ethyl methacrylate, 3-(N-methylamino)propyl acrylate, 3-(N-methylamino)propyl methacrylate, 3-(N-ethylamino)propyl acrylate, 3-(N-ethylamino)propyl methacrylate, 3-(N-n-propylamino)propyl acrylate, 3-(N-n-propylamino)propyl methacrylate, 3-(N-isopropylamino)propyl acrylate, 3-(N-isopropylamino)propyl methacrylate, 3-(N-tert-butylamino)propyl acrylate, 3-(N-tert-butylamino)propyl methacrylate, 3-(N,N-dimethylamino)propyl acrylate, 3-(N,N-dimethylamino)propyl methacrylate, 3-(N,N-diethylamino)propyl acrylate, 3-(N,N-diethylamino)propyl methacrylate, 3-(N,N-di-n-propylamino)propyl acrylate, 3-(N,N-di-n-propylamino)propyl methacrylate, 3-(N,N-diisopropylamino)propyl acrylate, and 3-(N,N-diisopropylamino)propyl methacrylate.

Examples of monomers FA which comprise at least one amido group are acrylamide, methacrylamide, N-methylacrylamide, N-methylmethacrylamide, N-ethylacrylamide, N-ethylmethacrylamide, N-n-propylacrylamide, N-n-propylmethacrylamide, N-isopropylacrylamide, N-isopropylmethacrylamide, N-tert-butylacrylamide, N-tert-butylmethacrylamide, N,N-dimethylacrylamide, N,N-dimethylmethacrylamide, N,N-diethylacrylamide, N,N-diethylmethacrylamide, N,N-di-n-propylacrylamide, N,N-di-n-propylmethacrylamide, N,N-diisopropylacrylamide, N,N-diisopropylmethacrylamide, N,N-di-n-butylacrylamide, N,N-di-n-butylmethacrylamide, N-(3-N′,N′-dimethylaminopropyl)methacrylamide, diacetoneacrylamide, N,N′-methylenebisacrylamide, N-(diphenylmethyl)acrylamide, N-cyclohexylacrylamide, and also N-vinylpyrrolidone and N-vinylcaprolactam.

Examples of monomers FA which comprise at least one ureido group are N,N′-divinylethyleneurea and 2-(1-imidazolin-2-onyl)ethyl methacrylate (available commercially, for example, as Norsocryl® 100 from Elf Atochem).

Examples of monomers FA which comprise at least one N-heterocyclic group are 2-vinylpyridine, 4-vinylpyridine, 1-vinylimidazole, 2-vinylimidazole, and N-vinylcarbazole.

Preference is given to using, as monomers FA, the following compounds: 2-vinylpyridine, 4-vinylpyridine, 2-vinylimidazole, 2-(N,N-dimethylamino)ethyl acrylate, 2-(N,N-dimethylamino)ethyl methacrylate, 2-(N,N-diethylamino)ethyl acrylate, 2-(N,N-diethylamino)ethyl methacrylate, 2-(N-tert-butylamino)ethyl methacrylate, N-(3-N′,N′-dimethylaminopropyl)methacrylamide, and 2-(1-imidazolin-2-onyl)ethyl methacrylate.

Depending on the pH of the aqueous reaction medium, it is possible for some or all of the aforementioned nitrogen-comprising monomers FA to be present in the N-protonated quaternary ammonium form.

Examples of monomers FA which have a quaternary alkylammonium structure on the nitrogen include 2-(N,N,N-trimethylammonium)ethyl acrylate chloride (available commercially, for example, as Norsocryl® ADAMQUAT MC 80 from Elf Atochem), 2-(N,N,N-trimethylammonium)ethyl methacrylate chloride (available commercially, for example, as Norsocryl® MADQUAT MC 75 from Elf Atochem), 2-(N-methyl-N,N-diethylammonium)ethyl acrylate chloride, 2-(N-methyl-N,N-diethylammonium)ethyl methacrylate chloride, 2-(N-methyl-N,N-dipropylammonium)ethyl acrylate chloride, 2-(N-methyl-N,N-dipropylammonium)ethyl methacrylate, 2-(N-benzyl-N,N-dimethylammonium)ethyl acrylate chloride (available commercially, for example, as Norsocryl® ADAMQUAT BZ 80 from Elf Atochem), 2-(N-benzyl-N,N-dimethylammonium)ethyl methacrylate chloride (available commercially, for example, as Norsocryl® MADQUAT BZ 75 from Elf Atochem), 2-(N-benzyl-N,N-diethylammonium)ethyl acrylate chloride, 2-(N-benzyl-N,N-diethylammonium)ethyl methacrylate chloride, 2-(N-benzyl-N,N-dipropylammonium)ethyl acrylate chloride, 2-(N-benzyl-N,N-dipropylammonium)ethyl methacrylate chloride, 3-(N,N,N-trimethylammonium)propyl acrylate chloride, 3-(N,N,N-trimethylammonium)propyl methacrylate chloride, 3-(N-methyl-N,N-diethylammonium)propyl acrylate chloride, 3-(N-methyl-N,N-diethylammonium)propyl methacrylate chloride, 3-(N-methyl-N,N-dipropylammonium)propyl acrylate chloride, 3-(N-methyl-N,N-dipropylammonium)propyl methacrylate chloride, 3-(N-benzyl-N,N-dimethylammonium)propyl acrylate chloride, 3-(N-benzyl-N,N-dimethylammonium)propyl methacrylate chloride, 3-(N-benzyl-N,N-diethylammonium)propyl acrylate chloride, 3-(N-benzyl-N,N-diethylammonium)propyl methacrylate chloride, 3-(N-benzyl-N,N-dipropylammonium)propyl acrylate chloride, and 3-(N-benzyl-N,N-dipropylammonium)propyl methacrylate chloride. It will be appreciated that it is also possible to use the corresponding bromides and sulfates instead of the chlorides specified.

Preference is given to using 2-(N,N,N-trimethylammonium)ethyl acrylate chloride, 2-(N,N,N-trimethylammonium)ethyl methacrylate chloride, 2-(N-benzyl-N,N-dimethylammonium)ethyl acrylate chloride, and 2-(N-benzyl-N,N-dimethylammonium)ethyl methacrylate chloride.

It will be appreciated that it is also possible to use mixtures of the aforementioned ethylenically unsaturated monomers FS and/or FA.

Advantageously in accordance with the invention, the ethylenically unsaturated monomer F used is a monomer mixture which comprises
from 50 to 99.9% by weight of esters of acrylic and/or methacrylic acid with alkanols having from 1 to 12 carbon atoms and/or styrene, or
from 50 to 99.9% by weight of styrene and butadiene, or
from 50 to 99.9% by weight of vinyl chloride and/or vinylidene chloride, or
from 40 to 99.9% by weight of vinyl acetate, vinyl propionate, vinyl esters of Versatic acid, vinyl esters of long-chain fatty acids and/or ethylene.

According to the invention, preference is given to ethylenically unsaturated monomers F or mixtures of monomers F which likewise have a low water solubility (analogously to solvents E).

The amount of ethylenically unsaturated monomers F used optionally in the first reaction stage is from 0 to 100% by weight, frequently from 30 to 90% by weight and often from 40 to 70% by weight, based in each case on the total amount of monomers F.

It is advantageous when the solvent E and/or the ethylenically unsaturated monomer F and their amounts in the first reaction stage are selected in such a way that the solubility of the solvent E and/or of the ethylenically unsaturated monomer F in the aqueous medium under reaction conditions of the first reaction stage is ≦50% by weight, ≦40% by weight, ≦30% by weight, ≦20% by weight or ≦10% by weight, based in each case on the total amount of solvent E and/or monomer F used optionally in the first reaction stage, and is thus present as a separate phase in the aqueous medium. The first reaction stage preferably proceeds in the presence of solvent E and/or monomer F, but especially preferably in the presence of monomer F and in the absence of solvent E.

Solvent E and/or monomer F are used in the first reaction stage especially when the diamine compound A and/or the dicarboxylic acid compound B have a good solubility in the aqueous medium under the reaction conditions of the first reaction stage, i.e. their solubility is >50 g/l or ≧100 g/l.

The process according to the invention proceeds advantageously when at least a portion of the diamine compound A, of the dicarboxylic acid compound B and also if appropriate of the solvent E and/or of the monomer F is present in the aqueous medium as a disperse phase having a mean droplet diameter of ≦1000 nm (what is known as an oil-in-water miniemulsion or a miniemulsion for short).

With particular advantage, the process according to the invention proceeds in the first reaction stage in such a way that at least a portion of diamine compound A, dicarboxylic acid compound B, dispersant D and, if appropriate, solvent E and/or monomer F is first introduced into a portion or the entirety of the water, then a disperse phase which comprises the diamine compound A, the dicarboxylic acid compound B and also, if appropriate, the solvent E and/or monomer F and has a mean droplet diameter of ≦1000 nm (miniemulsion) is obtained by means of suitable measures, and then the entirety of the enzyme C, and also the amounts which remain, if appropriate, of water, diamine compound A, dicarboxylic acid compound B, dispersant D and, if appropriate, solvent E, are added at reaction temperature to the aqueous medium. Frequently, ≧50% by weight, ≧60% by weight, ≧70% by weight, ≧80% by weight, ≧90% by weight or even the entireties of diamine compound A, dicarboxylic acid compound B, dispersant D and if appropriate solvent E are introduced into ≧50% by weight, ≧60% by weight, ≧70% by weight, ≧80% by weight, ≧90% by weight or even the entirety of the water, the disperse phase having a droplet diameter of ≦1000 nm is obtained, and then the entirety of the enzyme C and the amounts which remain, if appropriate, of water, diamine compound A, dicarboxylic acid compound B, dispersant D and if appropriate solvent E are added at reaction temperature to the aqueous medium. The enzyme C and the amounts which remain, if appropriate, of water, diamine compound A, dicarboxylic acid compound B, dispersant D and if appropriate solvent E may be added to the aqueous reaction medium discontinuously in one portion, discontinuously in several portions or continuously with uniform or varying mass flow rates.

Frequently, the entireties of diamine compound A, dicarboxylic acid compound B and if appropriate solvent E, and also at least a portion of the dispersant D, are introduced into the majority or entirety of the water and, after the miniemulsion has formed, the entirety of the enzyme C, if appropriate together with the remaining amounts of the water and of the dispersant D, are added at reaction temperature to the aqueous reaction medium.

The average size of the droplets of the disperse phase of the aqueous miniemulsion to be used advantageously in accordance with the invention can be determined by the principle of quasielastic dynamic light scattering (what is known as the z-average droplet diameter d_{z} of the unimodal analysis of the autocorrelation function), for example by means of a Coulter N4 Plus Particle Analyzer from Coulter Scientific Instruments. The measurements are undertaken on diluted aqueous miniemulsions whose content of nonaqueous constituents is approx. 0.01% by weight. The dilution is undertaken by means of water which had been saturated beforehand with the diamine compounds A, dicarboxylic acid compounds B and also if appropriate the low water solubility organic solvents E and/or ethylenically unsaturated monomers F present in the aqueous miniemulsion. The latter measure is intended to prevent the dilution from being accompanied by a change in the droplet diameter.

According to the invention, the values of d_{z} determined in this way for the miniemulsions are normally ≦700 nm, frequently ≦500 nm. According to the invention, the d_{z} range of from 100 nm to 400 nm or of from 100 nm to 300 nm is favorable. Normally, d_{z} of the aqueous miniemulsion to be used in accordance with the invention is ≧40 nm.

The general preparation of aqueous miniemulsions from aqueous macroemulsions is known to those skilled in the art (cf. P. L. Tang, E. D. Sudol, C. A. Silebi and M. S. EI-Aasser in Journal of Applied Polymer Science, Vol. 43, p. 1059 to 1066 [1991]).

For this purpose, high-pressure homogenizers, for example, may be employed. The fine dispersion of the components is achieved in these machines by a high localized energy input. Two variants have been found to be particularly useful for this purpose.

In the first variant, the aqueous macroemulsion is pressurized to above 1000 bar by means of a piston pump and is subsequently depressurized through a narrow slit. The action is based here on an interaction of high shear and pressure gradients and cavitation in the slit. An example of a high-pressure homogenizer which functions according to this principle is the Niro-Soavi high-pressure homogenizer model NS11001L Panda.

In the second variant, the pressurized aqueous macroemulsion is depressurized into a mixing chamber through two nozzles pointing toward one another. The fine-dispersing action is dependent here in particular on the hydrodynamic conditions in the mixing chamber. An example of a homogenizer of this type is the Microfluidizer model M 120 E from Microfluidics Corp. In this high-pressure homogenizer, the aqueous macroemulsion is compressed to pressures of up to 1200 atm by means of a pneumatically driven piston pump and is depressurized via an “interaction chamber”. In the “interaction chamber”, the jet of emulsion is divided in a microchannel system into two jets which are directed at one another at an angle of 180°. A further example of a homogenizer operating by this homogenization principle is the Nanojet model Expo from Nanojet Engineering GmbH. However, in the Nanojet, two homogenization valves which can be mechanically adjusted are installed in place of a fixed channel system.

In addition to the principles described above, the homogenization can also be carried out, for example, by use of ultrasound (for example Branson Sonifier II 450). The fine dispersion is based here on cavitation mechanisms. For the homogenization by means of ultrasound, the apparatus described in GB-A 22 50 930 and U.S. Pat. No. 5,108,654 is in principle also suitable. The quality of the aqueous miniemulsion obtained in the sonic field depends not only on the acoustic power introduced but also on other factors, for example the intensity distribution of the ultrasound in the mixing chamber, the residence time, the temperature and the physical properties of the substances to be emulsified, for example on the viscosity, the surface tension and the vapor pressure. The resulting droplet size depends, inter alia, on the concentration of the dispersant and on the energy introduced in the course of homogenization and can therefore be adjusted precisely by, for example, appropriate change in the homogenization pressure or the corresponding ultrasonic energy.

For the preparation of the aqueous miniemulsion used advantageously in accordance with the invention from conventional macroemulsions by means of ultrasound, the apparatus described in the prior German patent application DE-A 197 56 874 has been found to be particularly useful. This is an apparatus which comprises a reaction chamber or a flow-through reaction channel and at least one means of transmitting ultrasound waves into the reaction chamber or the flow-through reaction channel, the means for transmitting ultrasound waves being configured in such a way that the entire reaction chamber, or a section of the flow-through reaction channel, can be irradiated uniformly with ultrasound waves. For this purpose, the emitting surface of the means for transmitting ultrasound waves is configured in such a way that it corresponds essentially to the surface of the reaction chamber or, if the reaction chamber is a section of a flow-through reaction channel, extends essentially over the entire width of the channel, and in such a way that the depth of the reaction chamber in a direction essentially perpendicular to the emitting surface is less than the maximum depth of action of the ultrasound transmission means.

Here, the term “depth of the reaction chamber” refers essentially to the distance between the emitting surface of the ultrasound transmission means and the bottom of the reaction chamber.

Preference is given to reaction chamber depths up to 100 mm. The depth of the reaction chamber should advantageously be not more than 70 mm and particularly advantageously not more than 50 mm. The reaction chambers can in principle also have a very small depth, but with a view to a very low risk of blockage and easy cleaning and also a high product throughput, preference is given to reaction chamber depths which are significantly greater than, for example, the customary slit widths in high-pressure homogenizers and are usually above 10 mm. The depth of the reaction chamber is advantageously adjustable, for example by virtue of ultrasound transmission means being immersible to different depths into the casing.

In a first embodiment of this apparatus, the emitting surface of the means for transmitting ultrasound corresponds essentially to the surface of the reaction chamber. This embodiment is employed for the batchwise preparation of the miniemulsions used in accordance with the invention. In this apparatus, ultrasound can act over the entire reaction chamber. Turbulent flow is generated in the reaction chamber by the axial acoustic radiative pressure and this effects intensive transverse mixing.

In a second embodiment, such an apparatus has a flow-through cell. The casing is configured as a flow-through reaction channel which has an inlet and an outlet, the reaction chamber being a section of the flow-through reaction channel. The width of the channel is the channel dimension running essentially perpendicular to the flow direction. Here, the emitting surface covers the entire width of the flow channel transverse to the flow direction. The length of the emitting surface perpendicular to this width, i.e. the length of the emitting surface in the flow direction, defines the region of action of the ultrasound. In an advantageous variant of this first embodiment, the flow-through reaction channel has an essentially rectangular cross section. When a likewise rectangular ultrasound transmission means having appropriate dimensions is installed in one side of the rectangle, particularly effective and uniform sonication is achieved. Owing to the turbulent flow conditions existing in the ultrasonic field, it is, however, also possible to use, for example, a round transmission means without disadvantages. Moreover, a plurality of separate transmission means can be arranged in succession in the flow direction in place of a single ultrasound transmission means. In this case, both the emitting surfaces and the depth of the reaction chamber, i.e. the distance between the emitting surface and the bottom of the flow-through channel, can vary.

The means for transmitting ultrasound waves is particularly advantageously configured as a sonotrode whose end opposite the free emitting surface is coupled to an ultrasonic transducer. The ultrasound waves can, for example, be generated by exploiting the reverse piezoelectric effect. In this case, high-frequency electric oscillations (typically in the range from 10 to 100 kHz, preferably from 20 to 40 kHz) are generated with the aid of generators, converted to mechanical vibrations of the same frequency by means of a piezoelectric transducer and radiated by means of the sonotrode as transmission element into the medium to be sonicated.

The sonotrode is more preferably configured as a rod-shaped, axially emitting λ/2 (or multiples of λ/2) longitudinal oscillator. Such a sonotrode may, for example, be secured in an orifice of the casing by means of a flange provided at one of its nodes of oscillation. This allows the passage of the sonotrode into the casing to be configured in a pressure-tight manner, so that the sonication can also be carried out under elevated pressure in the reaction chamber. The oscillation amplitude of the sonotrode is preferably controllable, i.e. the oscillation amplitude established in each case is checked online and, if appropriate, automatically adjusted under closed-loop control. The current oscillation amplitude can be checked, for example, by a piezoelectric transducer mounted on the sonotrode or a strain gauge with downstream evaluation electronics.

In a further advantageous design of such apparatus, internals are provided within the reaction chamber to improve the flow and mixing performance. These internals may be, for example, simple baffle plates or a wide variety of porous bodies.

If required, the mixing may also be intensified by an additional stirrer. Advantageously, the temperature of the reaction chamber can be controlled.

It becomes clear from the above remarks that it is possible in accordance with the invention only to use those organic solvents E and/or ethylenically unsaturated monomers F whose solubility in the aqueous medium under reaction conditions is small enough to form solvent droplets and/or monomer droplets of ≦1000 nm as a separate phase with the specified amounts. In addition, the dissolution capacity of the solvent droplets and/or monomer droplets formed has to be large enough to take up at least portions, but preferably the entirety of the diamine compound A or dicarboxylic acid compound B.

It is important for the process according to the invention that, it is possible to use in the first reaction stage in addition to the diamine compound A and dicarboxylic acid compound B, an organic diol compound G, a hydroxycarboxylic acid compound H, an amino alcohol compound I, an aminocarboxylic acid compound K and/or an organic compound L which comprises at least 3 hydroxyl, primary or secondary amino and/or carboxyl groups per molecule. It is essential that the sum of the total amounts of individual compounds G, H, I, K and L is ≦50% by weight, preferably ≦40% by weight and especially preferably ≦30% by weight, and frequently ≧0.1% by weight or ≧1% by weight and often ≧5% by weight, based in each case on the sum of the total amounts of diamine compound A and dicarboxylic acid compound B.

The diol compounds G which find use in accordance with the invention are branched or linear alkanediols having from 2 to 18 carbon atoms, preferably from 4 to 14 carbon atoms, cycloalkanediols having from 5 to 20 carbon atoms, or aromatic diols.

Examples of suitable alkanediols are ethylene glycol, 1,2-propanediol, 1,3-propanediol, 1,2-butanediol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol, 2,4-dimethyl-2-ethyl-1,3-hexanediol, 2,2-dimethyl-1,3-propanediol (neopentyl glycol), 2-ethyl-2-butyl-1,3-propanediol, 2-ethyl-2-isobutyl-1,3-propanediol or 2,2,4-trimethyl-1,6-hexanediol. Especially suitable are ethylene glycol, 1,3-propanediol, 1,4-butanediol and 2,2-dimethyl-1,3-propanediol, 1,6-hexanediol or 1,12-dodecanediol.

Examples of cycloalkanediols are 1,2-cyclopentanediol, 1,3-cyclopentanediol, 1,2-cyclohexanediol, 1,3-cyclohexanediol, 1,4-cyclohexanediol, 1,2-cyclohexanedimethanol (1,2-dimethylolcyclohexane), 1,3-cyclohexanedimethanol (1,3-dimethylolcyclohexane), 1,4-cyclohexanedimethanol (1,4-dimethylolcyclohexane) or 2,2,4,4-tetramethyl-1,3-cyclobutanediol.

Examples of suitable aromatic diols are 1,4-dihydroxybenzene, 1,3-dihydroxybenzene, 1,2-dihydroxybenzene, bisphenol A (2,2-bis(4-hydroxyphenyl)propane), 1,3-dihydroxynaphthalene, 1,5-dihydroxynaphthalene or 1,7-dihydroxynaphthalene.

However, the diol compounds G used may also be polyetherdiols, for example diethylene glycol, triethylene glycol, polyethylene glycol (having ≧4 ethylene oxide units), propylene glycol, dipropylene glycol, tripropylene glycol, polypropylene glycol (having ≧4 propylene oxide units) and polytetrahydrofuran (polyTHF), in particular diethylene glycol, triethylene glycol and polyethylene glycol (having ≧4 ethylene oxide units). The polyTHF, polyethylene glycol or polypropylene glycol which find use are compounds whose number-average molecular weight (Mₙ) is generally in the range from 200 to 10 000 g/mol, preferably from 600 to 5000 g/mol.

Mixtures of the aforementioned diol compounds may also be used.

The hydroxycarboxylic acid compound H used can be hydroxycarboxylic acids and/or the lactones thereof. Examples include glycolic acid, D-, L-, D,L-lactic acid, 6-hydroxyhexanoic acid (6-hydroxycaproic acid), 3-hydroxybutyric acid, 3-hydroxyvaleric acid, 3-hydroxycaproic acid, p-hydroxybenzoic acid, the cyclic derivatives thereof such as glycolide (1,4-dioxane-2,5-dione), D-, L-, D,L-dilactide (3,6-dimethyl-1,4-dioxane-2,5-dione), ε-caprolactone, -butyrolactone, γ-butyrolactone, dodecanolide (oxacyclotridecan-2-one), undecanolide (oxacyclododecan-2-one) or pentadecanolide (oxacyclohexadecan-2-one). It will be appreciated that it is also possible to use mixtures of different hydroxycarboxylic acid compounds H.

The amino alcohol compound I used may in principle be any such compounds, but preferably C₂-C₁₂-aliphatic, C₅-C₁₀-cycloaliphatic or aromatic organic compounds which have only one hydroxyl group and a secondary or primary, but preferably a primary, amino group. Examples include 2-aminoethanol, 3-aminopropanol, 4-aminobutanol, 5-aminopentanol, 6-aminohexanol, 2-aminocyclopentanol, 3-aminocyclopentanol, 2-aminocyclohexanol, 3-aminocyclohexanol, 4-aminocyclohexanol and 4-aminomethylcyclohexanemethanol (1-methylol-4-aminomethylcyclohexane). It will be appreciated that it is also possible to use mixtures of the above amino alcohol compounds I.

It is also possible to use aminocarboxylic acid compounds K, which refers in the context of this document to aminocarboxylic acids and/or their corresponding lactam compounds, in addition to the diamine compound A and the dicarboxylic acid compound B. Examples include the naturally occurring aminocarboxylic acids such as valine, leucine, isoleucine, threonine, methionine, phenylalanine, tryptophan, lysine, alanine, arginine, aspartic acid, cysteine, glutamic acid, glycine, histidine, proline, serine, tyrosine, asparagine or glutamine, and also 3-aminopropionic acid, 4-aminobutyric acid, 5-aminovaleric acid, 6-aminocaproic acid, 7-aminoenanthic acid, 8-aminocaprylic acid, 9-aminopelargonic acid, 10-aminocapric acid, 11-aminoundecanoic acid, 12-aminolauric acid and the lactams α-propiolactam, γ-butyrolactam, δ-valerolactam, ε-caprolactam, 7-enantholactam, 8-caprylolactam, 9-pelargolactam, 10-decanolactam, 11-undecanolactam or ω-laurolactam. Preference is given to ε-caprolactam and ω-laurolactam. It will be appreciated that mixtures of the aforementioned aminocarboxylic acid compounds K may also be used.

A further component which may be used optionally in the process according to the invention is an organic compound L which comprises at least 3 hydroxyl, primary or secondary amino and/or carboxyl groups per molecule. Examples include tartaric acid, citric acid, malic acid, trimethylolpropane, trimethylolethane, pentaerythritol, polyethertriols, glycerol, sugar (for example glucose, mannose, fructose, galactose, glucosamine, sucrose, lactose, trehalose, maltose, cellobiose, gentianose, kestose, maltotriose, raffinose, trimesic acid (1,3,5-benzenetricarboxylic acid and the esters or anhydrides thereof, trimellitic acid (1,2,4-benzenetricarboxylic acid and the esters or anhydrides thereof), pyromellitic acid (1,2,4,5-benzenetetracarboxylic acid and the esters or anhydrides thereof, 4-hydroxyisophthalic acid, diethylenetriamine, dipropylenetriamine, bishexamethylenetriamine, N,N′-bis(3-aminopropyl)ethylenediamine, diethanolamine or triethanolamine. The aforementioned compounds L are capable by virtue of their at least 3 hydroxyl, primary or secondary amino and/or carboxyl groups per molecule of being incorporated simultaneously into at least 2 polyamide chains, which is why compounds L have a branching or crosslinking action in the polyamide formation. The higher the content of compound L, and the more amino, hydroxyl and/or carboxyl groups are present per molecule, the higher the degree of branching/crosslinking in the polyamide formation. It will be appreciated that it is also possible in this context to use mixtures of compounds L.

According to the invention, it is also possible to use mixtures of organic diol compound G, hydroxycarboxylic acid compound H, amino alcohol compound I, aminocarboxylic acid compound K and/or organic compound L which comprises at least 3 hydroxyl, primary or secondary amino and/or carboxyl groups per molecule.

When, in accordance with the invention, at least one of the aforementioned compounds G to L is also used in the first reaction stage in addition to the diamine compound A and the dicarboxylic acid compound B, it has to be ensured that the amounts of the compounds A and B and also G to L are selected such that the ratio of equivalents of the carboxyl groups and/or derivatives thereof (from the individual compounds B, H, K and L) to the sum of amino and/or hydroxyl groups and/or derivatives thereof (from the individual compounds A, G, H, I, K and L) is from 0.5 to 1.5, generally from 0.8 to 1.3, frequently from 0.9 to 1.1 and often from 0.95 to 1.05. It is particularly favorable when the ratio of equivalents is 1, i.e. just as many amino and hydroxyl groups are present as carboxyl groups or groups derived therefrom. For a better understanding, it should be pointed out that the dicarboxylic acid compounds B (free acid, ester, halide or anhydride) comprise 2 equivalents of carboxyl groups, the hydroxycarboxylic acid compounds H or the aminocarboxylic acid compounds K comprise in each case one equivalent of carboxyl groups and the organic compounds L have as many equivalents of carboxyl groups as they comprise carboxyl groups per molecule. Correspondingly, the diamine compounds A comprise 2 equivalents of amino groups, the diol compounds G comprise 2 equivalents of hydroxyl groups, the hydroxycarboxylic acid compounds H comprise one hydroxyl group equivalent, the amino carboxylic acid compounds K comprise one amino group equivalent, and the organic compounds L comprise as many equivalents of hydroxyl and amino groups as they comprise hydroxyl and amino groups in the molecule.

It is self-evident for the process according to the invention that the enzymes C are selected so as to be compatible especially with the diamine compounds A, dicarboxylic acid compounds B, organic diol compounds G, hydroxycarboxylic acid compounds H, amino alcohol compounds I, aminocarboxylic acid compounds K, organic compounds L which comprise at least 3 hydroxyl, primary or secondary amino, carboxyl groups per molecule used, and the dispersants D, the solvents E and/or the ethylenically unsaturated monomers F, and not to be deactivated by them. Which compounds A and B and also D to L can be used for a certain enzyme C is known or can be determined by those skilled in the art in simple preliminary experiments.

When one of the aforementioned compounds G, H, I, K and/or L is used in addition to the diamine compound A and the dicarboxylic acid compound B, the first reaction stage of the process according to the invention advantageously proceeds in such a way that at least a portion of diamine compound A, dicarboxylic acid compound B, compound G, H, I, K and/or L, dispersant D and if appropriate solvent E and/or ethylenically unsaturated monomer F is first introduced into at least a portion of the water, then a disperse phase which comprises the diamine compound A, the dicarboxylic acid compound B, the compound G, H, I, K and/or L and also, if appropriate, the solvent E and/or the ethylenically unsaturated monomer F and has a mean droplet diameter of ≦1000 nm (miniemulsion) is obtained by means of suitable measures, and then the entirety of enzyme C, and also the amounts which remain, if appropriate, of diamine compound A, dicarboxylic acid compound B, compound G, H, I, K and/or L and solvent E, are added at reaction temperature to the aqueous medium. Frequently, ≧50% by weight, ≧60% by weight, ≧70% by weight, ≧80% by weight, ≧90% by weight or even the entireties of diamine compound A, dicarboxylic acid compound B, compound G, H, I, K and/or L, dispersant D and also, if appropriate, solvent E are introduced into ≧50% by weight, ≧60% by weight, ≧70% by weight, ≧80% by weight, ≧90% by weight or even the entirety of the water, then the disperse phase having a droplet diameter of ≦1000 nm is obtained, and then the entirety of enzyme C and the amounts which remain, if appropriate, of diamine compound A, dicarboxylic acid compound B, compound G, H, I, K and/or L, and also solvent E are added at reaction temperature to the aqueous medium. The enzyme C and the amounts which remain, if appropriate, of diamine compound A, dicarboxylic acid compound B, compound G, H, I, K and/or L, and also solvent E may be added to the aqueous reaction medium separately or together, discontinuously in one portion, discontinuously in several portions or continuously with uniform or varying mass flow rates.

The first reaction stage of the process according to the invention proceeds generally at a reaction temperature of from 20 to 90° C., often from 35 to 60° C. and frequently from 45 to 55° C., at a pressure (absolute values) of generally from 0.8 to 10 bar, preferably from 0.9 to 2 bar and in particular at 1 atm (=1.01 bar=atmospheric pressure).

It is further advantageous when the aqueous reaction medium in the first reaction stage has a pH at room temperature (20 to 25° C.) of ≧2 and ≦11, frequently ≧3 and ≦9 and often ≧6 and ≦8. In particular, such a pH (range) is established in the aqueous reaction medium at which the enzyme C has optimal action. Which pH (range) this is known to or can be determined by those skilled in the art in a few preliminary experiments. The appropriate measures for adjusting the pH, i.e. addition of appropriate amounts of acid, for example sulfuric acid, bases, for example aqueous solutions of alkali metal hydroxides, in particular sodium hydroxide or potassium hydroxide, or buffer substances, for example potassium dihydrogenphosphate/disodium hydrogenphosphate, acetic acid/sodium acetate, ammonium hydroxide/ammonium chloride, potassium dihydrogenphosphate/sodium hydroxide, borax/hydrochloric acid, borax/sodium hydroxide or tris(hydroxymethyl)aminomethane/hydrochloric acid, are familiar to those skilled in the art.

For the process according to the invention, the water used is typically clear and frequently has drinking water quality. However, the water used for the process according to the invention is advantageously deionized water, and in the first reaction stage sterile deionized water. The amount of water in the first reaction stage is selected in such a way that the aqueous polyamide dispersion formed in accordance with the invention has a water content of ≧30% by weight, frequently ≧50 and ≦99% by weight or ≧65 and ≦95% by weight and often ≧70 and ≦90% by weight, based in each case on the aqueous polyamide dispersion, corresponding to a polyamide solids content of ≦70% by weight, frequently ≧1 and ≦50% by weight or ≧5 and ≦35% by weight and often ≧10 and ≦30% by weight. It should also be mentioned here that the process according to the invention, both in the first and in the second reaction stage, is carried out advantageously under oxygen-free inert gas atmosphere, for example under nitrogen or argon atmosphere.

Advantageously in accordance with the invention, an assistant (deactivator) which is capable of deactivating the enzyme C used in accordance with the invention (i.e. of destroying or of inhibiting the catalytic action of the enzyme C) is added to the aqueous polyamide dispersion of the first reaction stage after or at the end of the enzymatically catalyzed polymerization reaction. The deactivators used may be any compounds which are capable of deactivating the particular enzyme C. The deactivators used may frequently in particular be complexes, for example nitrilotriacetic acid or ethylenediaminetetraacetic acid or alkali metal salts thereof, or anionic emulsifiers, for example sodium dodecylsulfate. Their amount is typically just enough to deactivate the particular enzyme C. It is frequently also possible to deactivate the enzymes C used by heating the aqueous polyamide dispersion to temperatures of ≧95° C. or ≧100° C., in the course of which inert gas is generally injected under pressure to suppress a boiling reaction. It will be appreciated that it is also possible to deactivate certain enzymes C by changing the pH of the aqueous reaction medium.

The polyamides obtainable by the process according to the invention in the first reaction stage may have glass transition temperatures of from −70 to +200° C. Depending on the end use, polyamides are frequently required whose glass transition temperatures lie within particular ranges. Suitable selection of the components A and B and also G to L used in the process according to the invention makes it possible for those skilled in the art to selectively prepare polyamides whose glass transition temperatures lie within the desired range.

The glass transition temperature T_{g} means the limiting value of the glass transition temperature, the glass transition temperature approaching the limiting value with increasing molecular weight according to G. Kanig (Kolloid-Zeitschrift & Zeitschrift für Polymere, Vol. 190, page 1, equation 1). The glass transition temperature is determined by the DSC process (Differential Scanning Calorimetry, 20 K/min, midpoint measurement, DIN 53 765).

The polyamide particles of the aqueous polyamide dispersions obtainable by the process according to the invention have average particle diameters which are generally between 10 and 1000 nm, frequently between 50 and 700 nm and often between 100 and 500 nm [the values reported are the cumulant z-average values, determined by quasielastic light scattering (ISO standard 13 321)].

The polyamides obtainable in the first reaction stage by the process according to the invention generally have a weight-average molecular weight in the range from ≧2000 to ≦1 000 000 g/mol, often from ≧3000 to ≦500 000 g/mol or from ≧5000 to ≦100 000 g/mol and frequently from ≧5000 to ≦50 000 g/mol or from ≧6000 to ≦30 000 g/mol. The weight-average molecular weights are determined by means of gel permeation chromatography based on DIN 55672-1.

It is essential to the process that, in a second reaction stage, an ethylenically unsaturated monomer F is free-radically polymerized in the aqueous medium which comprises the polyamide formed in the first reaction stage. This polymerization is effected advantageously under the conditions of a free-radically initiated aqueous emulsion polymerization. This method has been described many times before and is therefore sufficiently well known to those skilled in the art [cf., for example, Encyclopedia of Polymer Science and Engineering, Vol. 8, pages 659 to 677, John Wiley & Sons, Inc., 1987; D. C. Blackley, Emulsion Polymerisation, pages 155 to 465, Applied Science Publishers, Ltd., Essex, 1975; D. C. Blackley, Polymer Latices, 2^{nd} Edition, Vol. 1, pages 33 to 415, Chapman & Hall, 1997; H. Warson, The Applications of Synthetic Resin Emulsions, pages 49 to 244, Ernest Benn, Ltd., London, 1972; D. Diederich, Chemie in unserer Zeit 1990, 24, pages 135 to 142, Verlag Chemie, Weinheim; J. Piirma, Emulsion Polymerisation, pages 1 to 287, Academic Press, 1982; F. Hölscher, Dispersionen synthetischer Hochpolymerer, pages 1 to 160, Springer-Verlag, Berlin, 1969 and the patent DE-A 40 03 422]. The free-radically initiated aqueous emulsion polymerization is effected typically in such a way that the ethylenically unsaturated monomers, generally with use of dispersants, are distributed dispersed in an aqueous medium and polymerized by means of at least one water-soluble free-radical polymerization initiator at polymerization temperature.

In order to obtain stable aqueous polymer dispersions in the second reaction step, the dispersant D and its amount have to be such that it is capable of stabilizing, as disperse phases in the aqueous medium, both the polyamide particles formed in the first reaction stage and the ethylenically unsaturated monomer F used for the polymerization of the second reaction stage in the form of monomer droplets, and also the polymer particles formed in the free-radical polymerization reaction. The dispersant D of the second reaction stage may be identical to that of the first reaction stage. However, it is also possible that a further dispersant D is added in the second reaction stage. It is also possible that the entirety of dispersant D has already been added to the aqueous medium in the first reaction stage. However, it is also possible that portions of dispersant D are added to the aqueous medium in the second reaction stage before, during or after, in particular before or during, the free-radical polymerization. This is the case in particular when, in the first reaction stage, different or smaller amounts of dispersant D were used or, in the second reaction stage, a portion or the entirety of the ethylenically unsaturated monomer F is used in the form of an aqueous monomer emulsion. Which dispersant D and in what amount it is used additionally advantageously in the second reaction stage is known to or can be determined by those skilled in the art in simple preliminary experiments. Frequently, the amount of dispersant D added in the first reaction stage is ≧1 and ≦100% by weight, ≧20 and ≦90% by weight or ≧40 and ≦70% by weight, and, in the second reaction stage, accordingly ≧0 and ≦99% by weight, ≧10 and ≦80% by weight, or ≧30 and ≦60% by weight, based in each case on the total amount of dispersant used in the process according to the invention.

The emulsifiers used with preference as the dispersant D are used advantageously in a total amount of from 0.005 to 20% by weight, preferably from 0.01 to 10% by weight, in particular from 0.1 to 5% by weight, based in each case on the sum of the total amounts of diamine compound A, dicarboxylic acid compound B and ethylenically unsaturated monomer F.

The total amount of the protective colloids used as the dispersant D in addition to or instead of the emulsifiers is often from 0.1 to 10% by weight and frequently from 0.2 to 7% by weight, based in each case on the sum of the total amounts of diamine compound A, dicarboxylic acid compound B and ethylenically unsaturated monomer F.

However, preference is given to using emulsifiers, especially nonionic emulsifiers, as the sole dispersant D.

The entire amount of the water used in the process according to the invention may be used in the first reaction stage. However, it is also possible to add portions of water in the first and in the second reaction stage. Portions of water are added in the second reaction stage in particular when ethylenically unsaturated monomers F are added in the second reaction stage in the form of an aqueous monomer emulsion and the free-radical initiator is added in the form of a corresponding aqueous solution or aqueous dispersion of the free-radical initiator. In general, the total amount of water is selected in such a way that the aqueous polymer dispersion formed in accordance with the invention has a water content of ≧30% by weight, frequently ≧40 and ≦99% by weight or ≧45 and ≦95% by weight, and often ≧50 and ≦90% by weight, based in each case on the aqueous polymer dispersion, corresponding to a polymer solids content of ≦70% by weight, frequently ≧1 and ≦60% by weight or ≧5 and ≦55% by weight, and often ≧10 and ≦50% by weight. Frequently, the amount of water added in the first reaction stage is ≧10 and ≦100% by weight, ≧40 and ≦90% by weight or ≧60 and ≦80% by weight, and, in the second reaction stage, accordingly ≧0 and ≦90% by weight, ≧10 and ≦60% by weight or ≧20 and ≦40% by weight, based in each case on the total amount of water used in the process according to the invention.

The entirety of monomers F used in the process according to the invention may be used either in the first or in the second reaction stage. However, it is also possible to add portions of monomers F in the first and in the second reaction stage. Portions or the entirety of monomers F are added in the second reaction stage especially in the form of an aqueous monomer emulsion. The total amount of monomers F is generally selected such that the aqueous polymer dispersion formed in accordance with the invention has a solids content of polymer (=sum of polyamide of the first reaction stage and polymer obtained by polymerization of the ethylenically unsaturated monomer F in the second reaction stage) of ≦70% by weight, frequently ≧1 and ≦60% by weight or ≧5 and ≦55% by weight, and often ≧10 and ≦50% by weight. Frequently, the amount of monomers F added in the first reaction stage is ≧0 and ≦100% by weight, ≧20 and ≦90% by weight or ≧40 and ≦70% by weight, and, in the second reaction stage, accordingly ≧0 and ≦100% by weight, ≧10 and ≦80% by weight or ≧30 and ≦60% by weight, based in each case on the total amount of monomers F.

According to the invention, the quantitative ratio of the sum of the total amounts of diamine compound A and dicarboxylic acid compound B to the total amount of ethylenically unsaturated monomers F is generally from 1:99 to 99:1, preferably from 1:9 to 9:1 and advantageously from 1:5 to 5:1.

Advantageously, at least a portion, but preferably the entirety, of monomers F is used in the first reaction stage. This has the advantage that the polyamide particles formed in the first reaction stage comprise dissolved monomers F or are swollen with them, or the polyamide is dissolved or dispersed in the droplets of the monomers F. Both have advantageous effects on the formation of polymer (hybrid) particles which are formed from the polyamide of the first reaction stage and the polymer of the second reaction stage.

The polymers obtainable from the monomers F in the second reaction stage by the process according to the invention may have glass transition temperatures of from −70 to +150° C. Depending on the planned end use of the aqueous polymer dispersion, polymers are frequently required whose glass transition temperatures lie within certain ranges. Suitable selection of the monomers F used in the process according to the invention makes it possible for those skilled in the art to selectively prepare polymers whose glass transition temperatures lie within the desired range.

According to Fox (T. G. Fox, Bull. Am. Phys. Soc. 1956 [Ser. II] 1, page 123 and according to Ullmann's Encyclopedia of Industrial Chemistry, Vol. 19, page 18, 4^{th} edition, Verlag Chemie, Weinheim, 1980), a good approximation of the glass transition temperature of at most slightly crosslinked copolymers is:

1/T_{g}=x¹/T_{g}¹+x²/T_{g}²+ . . . xⁿ/T_{g}ⁿ

where x¹, x², . . . xⁿ are the mass fractions of the monomers 1, 2, . . . n, and T_{g}¹, T_{g}², . . . T_{g}ⁿ are the glass transition temperatures of the polymers formed in each case only from one of the monomers 1, 2, . . . n in degrees Kelvin. The T_{g} values for the homopolymers of most monomers are known and are listed, for example, in Ullmann's Encyclopedia of Industrial Chemistry, 5^{th} Ed., Vol. A21, page 169, Verlag Chemie, Weinheim, 1992; further sources of glass transition temperatures of homopolymers are, for example, J. Brandrup, E. H. Immergut, Polymer Handbook, 1^{st} Ed., J. Wiley, New York, 1966; 2^{nd} Ed. J. Wiley, New York, 1975 and 3^{rd} Ed. J. Wiley, New York, 1989.

A characteristic feature of the process according to the invention is that the free-radically induced polymerization in the second reaction stage can be triggered by using either what are referred to as water-soluble or what are referred to as oil-soluble free-radical initiators. Water-soluble free-radical initiators are generally understood to be all free-radical initiators which are used typically in free-radically initiated aqueous emulsion polymerization, while oil-soluble free-radical initiators refer to all of those free-radical initiators which those skilled in the art use typically in free-radically initiated solution polymerization. In the context of this document, water-soluble free-radical initiators should be understood to mean all of those free-radical initiators which have a solubility of ≧1% by weight in deionized water at 20° C. and atmospheric pressure, while oil-soluble free-radical initiators should be understood to mean all of those free-radical initiators which have a solubility of ≦1% by weight under the aforementioned conditions. Frequently, water-soluble free-radical initiators have a water solubility under the aforementioned conditions of ≧2% by weight, ≧5% by weight or ≧10% by weight, while oil-soluble free-radical initiators frequently have a water solubility of ≦0.9% by weight, ≦0.8% by weight, ≦0.7% by weight, ≦0.6% by weight, ≦0.5% by weight, ≦0.4% by weight, ≦0.3% by weight, ≦0.2% by weight or ≦0.1% by weight.

The water-soluble free-radical initiators may, for example, either be peroxides or azo compounds. It will be appreciated that redox initiator systems may also be used. The peroxides used may in principle be inorganic peroxides such as hydrogen peroxide or peroxodisulfates such as the mono- or dialkali metal or ammonium salts of peroxodisulfuric acid, for example their mono- and disodium, -potassium or -ammonium salts, or organic peroxides such as alkyl hydroperoxides, for example tert-butyl, p-menthyl or cumyl hydroperoxide. The azo compounds which find use are essentially 2,2′-azobis(isobutyronitrile), 2,2′-azobis(2,4-dimethylvaleronitrile) and 2,2′-azobis(amidinopropyl) dihydrochloride (AIBA, corresponds to V-50 from Wako Chemicals). The oxidizing agents used for redox initiator systems are essentially the abovementioned peroxides. Corresponding reducing agents may be sulfur compounds having a low oxidation state, such as alkali metal sulfites, for example potassium and/or sodium sulfite, alkali metal hydrogensulfites, for example potassium and/or sodium hydrogensulfite, alkali metabisulfites, for example potassium and/or sodium metabisulfite, formaldehydesulfoxylates, for example potassium and/or sodium formaldehydesulfoxylate, alkali metal salts, specifically potassium and/or sodium salts of aliphatic sulfinic acids and alkali metal hydrogensulfides, for example potassium and/or sodium hydrogensulfide, salts of polyvalent metals, such as iron(II) sulfate, iron(II) ammonium sulfate, iron(II) phosphate, enediols such as dihydroxymaleic acid, benzoin and/or ascorbic acid, and also reducing saccharides such as sorbose, glucose, fructose and/or dihydroxyacetone.

The water-soluble free-radical initiators used are preferably a mono- or dialkali metal or ammonium salt of peroxodisulfuric acid, for example dipotassium peroxydisulfate, disodium peroxydisulfate or diammonium peroxydisulfate. It will be appreciated that it is also possible to use mixtures of the aforementioned water-soluble free-radical initiators.

Examples of oil-soluble free-radical initiators include dialkyl or diaryl peroxides such as di-tert-amyl peroxide, dicumyl peroxide, bis(tert-butylperoxyisopropyl)benzene, 2,5-bis(tert-butylperoxy)-2,5-dimethylhexane, tert-butylcumene peroxide, 2,5-bis(tert-butylperoxy)-2,5-dimethyl-3-hexene, 1,1-bis(tert-butylperoxy)-3,3,5-trimethylcyclohexane, 1,1-bis(tert-butylperoxy)cyclohexane, 2,2-bis(tert-butylperoxy)butane or di-tert-butylperoxide, aliphatic and aromatic peroxy esters such as cumyl peroxyneodecanoate, 2,4,4-trimethyl-2-pentyl peroxyneodecanoate, tert-amyl peroxyneodecanoate, tert-butyl peroxyneodecanoate, tert-amyl peroxypivalate, tert-butyl peroxypivalate, tert-amyl peroxy-2-ethylhexanoate, tert-butyl peroxy-2-ethylhexanoate, tert-butyl peroxy-diethylacetate, 1,4-bis(tert-butylperoxy)cyclohexane, tert-butyl peroxyisobutanoate, tert-butyl peroxy-3,5,5-trimethylhexanoate, tert-butyl peroxyacetate, tert-amyl peroxybenzoate or tert-butyl peroxybenzoate, dialkanoyl or dibenzoyl peroxides such as diisobutanoyl peroxide, bis(3,5,5-trimethylhexanoyl) peroxide, dilauroyl peroxide, didecanoyl peroxide, 2,5-bis(2-ethylhexanoylperoxy)-2,5-dimethylhexane or dibenzoyl peroxide, and also peroxycarbonates such as bis(4-tert-butylcyclohexyl)peroxydicarbonate, bis(2-ethylhexyl) peroxydicarbonate, di-tert-butyl peroxydicarbonate, dicetyl peroxydicarbonate, dimyristyl peroxydicarbonate, tert-butyl peroxyisopropylcarbonate or tert-butyl peroxy-2-ethylhexylcarbonate.

The oil-soluble free-radical initiator used is preferably a compound selected from the group comprising tert-butyl peroxy-2-ethylhexanoate (Trigonox® 21), tert-amyl peroxy-2-ethylhexanoate, tert-butyl peroxybenzoate (Trigonox® C), tert-amyl peroxybenzoate, tert-butyl peroxyacetate, tert-butyl peroxy-3,5,5-trimethylhexanoate (Trigonox® 42 S), tert-butyl peroxyisobutanoate, tert-butyl peroxydiethylacetate, tert-butyl peroxypivalate, tert-butyl peroxyisopropylcarbonate (Trigonox® BPIC) and tert-butyl peroxy-2-ethylhexylcarbonate (Trigonox® 117). It will be appreciated that it is also possible to use mixtures of the aforementioned oil-soluble free radical initiators.

Particular preference is given to using water-soluble free-radical initiators.

The total amount of free-radical initiator used is from 0.01 to 5% by weight, frequently from 0.5 to 3% by weight and often from 1 to 2% by weight, based in each case on the total amount of monomers F.

A possible reaction temperature for the free-radical polymerization reaction of the second reaction stage, depending on factors including the free-radical initiator used, is the entire range from 0 to 170° C. The temperatures employed are generally from 50 to 120° C., frequently from 60 to 110° C. and often from ≧70 to 100° C. The free-radical polymerization reaction of the second reaction stage may be carried out at a pressure less than, equal to or greater than 1 atm (absolute), and the polymerization temperature may exceed 100° C. and be up to 170° C. Preference is given to polymerizing volatile monomers such as ethylene, butadiene or vinyl chloride under elevated pressure. In this case, the pressure may be 1.2, 1.5, 2, 5, 10, 15 bar or take on even higher values. When emulsion polymerizations are carried out under reduced pressure, pressures of 950 mbar, frequently of 900 mbar and often 850 mbar (absolute) are established. Advantageously, the free-radical polymerization reaction is carried out under an inert gas atmosphere at atmospheric pressure.

The free-radical polymerization of the second reaction stage is effected generally up to a conversion of the monomers F of ≧90% by weight, advantageously ≧95% by weight and preferably ≧98% by weight.

With particular advantage, the process according to the invention proceeds in such a way that, in the first reaction stage, at least a portion of diamine compound A, dicarboxylic acid compound B, dispersant D and, if appropriate, solvent E and/or ethylenically unsaturated monomer F is first introduced into at least a portion of the water, then a disperse phase which comprises the diamine compound A, dicarboxylic acid compound B and also, if appropriate, the solvent E and/or if appropriate the ethylenically unsaturated monomer F and has a mean droplet diameter of ≦1000 nm is obtained by means of suitable measures, and then the entirety of the enzyme C, and also the amounts which remain, if appropriate, of diamine compound A, dicarboxylic acid compound B and solvent E, are added at reaction temperature to the aqueous medium, and, on completion of polyamide formation, in the second reaction stage, the amounts which remain, if appropriate, of water, dispersant D and/or ethylenically unsaturated monomer F and the entirety of a free-radical initiator are added. The amounts which remain, if appropriate, of water, dispersant D and/or ethylenically unsaturated monomer F and also the entirety of a free-radical initiator may be added separately or together, in one portion, discontinuously in several portions or continuously with uniform or varying mass flow rates.

The aqueous polymer dispersions obtainable by the process according to the invention are suitable advantageously as components in adhesives, sealants, polymer renders, papercoating slips, printing inks, cosmetic formulations and paints, for finishing leather and textiles, for fiber binding, and for modifying mineral binders or asphalt.

It is also of significance that the aqueous polymer dispersions obtainable in accordance with the invention can be converted by drying to the corresponding polymer powders. Corresponding drying methods, for example freeze-drying or spray-drying, are known to those skilled in the art.

The polymer powders obtainable in accordance with the invention can be used advantageously as a pigment, filler in polymer formulations, as a component in adhesives, sealants, polymer renders, papercoating slips, printing inks, cosmetic formulations, powder coatings and paints, for finishing leather and textiles, for fiber binding, and for modifying mineral binders or asphalt.

The process according to the invention opens up a simple and inexpensive route to novel aqueous polymer dispersions which combine both the product properties of the polyamides and those of the polymers.

The nonrestrictive example which follows will illustrate the invention.

### EXAMPLE

Under a nitrogen atmosphere, 1.4 g (5.8 mmol) of 3,3′-dimethyl-4,4′-diaminodicyclohexylmethane (Laromin® C260, commercial product from BASF AG), 1.55 g (5.8 mmol) of diethyl sebacate (98% by weight, from Sigma-Aldrich Inc.), 1.85 g (17.8 mmol) of styrene and 0.24 g of hexadecane were mixed homogeneously at room temperature (20 to 25° C.) by stirring by means of a magnetic stirrer. With stirring, a homogeneous solution of 0.24 g of Lutensol® AT 50 (nonionic emulsifier, commercial product from BASF AG) and 23.8 g of a buffer solution (with a pH of 6.87, comprising 0.025 mol/l of potassium dihydrogenphosphate (KH₂PO₄) and 0.025 mol/l of disodium hydrogenphosphate (Na₂HPO₄) in deionized water) were added to this mixture. Subsequently, the resulting heterogeneous mixture was stirred with a magnetic stirrer at 60 revolutions per minute (rpm) for 10 minutes, then transferred, likewise under nitrogen, into an 80 ml conical-shoulder vessel and stirred by means of an Ultra-Turrax T25 unit (from Janke & Kunkel GmbH & Co. KG) at 20 500 rpm for 30 seconds. Afterward, the resulting liquid heterogeneous mixture was converted to droplets having a mean droplet diameter of ≦1000 nm by subjecting it to ultrasound treatment by means of an ultrasound probe (70 W; UW 2070 unit from Bandelin electronic GmbH & Co. KG) for 3 minutes. A homogeneous enzyme mixture prepared from 0.24 g of lipase from Candida antarctica type B (commercial product from Fluka AG), 0.14 g of Lutensol® AT 50 and 14.4 g of the aforementioned buffer solution was then added under nitrogen in one portion to the thus prepared miniemulsion, then the resulting mixture was heated to 50° C. with stirring and the mixture was stirred at this temperature under a nitrogen atmosphere for 20 hours.

For enzyme deactivation, 0.05 g of sodium dodecylsulfate was subsequently added with stirring, and the aqueous polyamide dispersion was stirred at 50° C. for a further 30 minutes. Subsequently, a solution consisting of 0.03 g of sodium peroxodisulfate and 0.27 g of deionized water was added under a nitrogen atmosphere with stirring to the resulting aqueous polyamide dispersion, the polymerization mixture was heated to 80° C., the mixture was stirred at this temperature for 2 hours, and then the resulting aqueous polymer dispersion was cooled to room temperature.

Approx. 44 g of an aqueous polymer dispersion having a solids content of 11.5% by weight were obtained. The mean particle size was determined to be 270 nm. The glass transition temperature was determined to be 55° C. (polyamide) and approx. 100° C. (polystyrene). The polymer also had melting points at 155° C. and 220° C.

The solids content was determined by drying a defined amount of the aqueous polymer dispersion (approx. 5 g) to constant weight at 180° C. in a drying cabinet. In each case, two separate analyses were carried out. The value reported in the example constitutes the mean value of the two measurements.

The mean particle diameter of the polymer particles was determined by dynamic light scattering on a from 0.005 to 0.01 percent by weight aqueous polymer dispersion at 23° C. by means of an Autosizer IIC from Malvern Instruments, England. The mean diameter of the cumulant evaluation (cumulant z-average) of the measured autocorrelation function (ISO standard 13321) is reported.

The glass transition temperature and the melting point were determined according to DIN 53765 by means of a DSC820 unit, TA8000 series from Mettler-Toledo Intl. Inc.

1: A process for preparing an aqueous polymer dispersion, which comprises reacting, in an aqueous medium, in a first reaction stage,
a) a diamine compound A and
b) a dicarboxylic acid compound B,
in the presence
c) of an enzyme C which catalyzes a polycondensation reaction of diamine compound A and dicarboxylic acid compound B, and
d) of a dispersant D,
and optionally,
e) of a low water solubility organic solvent E and/or
f) of an ethylenically unsaturated monomer F,
to give a polyamide, and thereafter, in the presence of the polyamide, in a second reaction stage, free-radically polymerizing an ethylenically unsaturated monomer F.

2: The process according to claim 1, wherein, in the first reaction stage, at least a portion of the diamine compound A, of the dicarboxylic acid compound B, of the solvent E and/or of the ethylenically unsaturated monomer F is present in the aqueous medium as a disperse phase having a mean droplet diameter of ≦1000 nm.

3: The process according to claim 2, wherein at least a portion of diamine compound A, dicarboxylic acid compound B, dispersant D and optionally, solvent E and/or ethylenically unsaturated monomer F is first introduced into at least a portion of water, then a disperse phase which comprises the diamine compound A, the dicarboxylic acid compound B and optionally, the solvent E and/or the ethylenically unsaturated monomer F and has a mean droplet diameter of ≦1000 nm is obtained by means of suitable measures, and then the entirety of the enzyme C, and also the amounts which remain of diamine compound A, dicarboxylic acid compound B and solvent E, are added at reaction temperature to the aqueous medium.

4: The process according to claim 1, wherein the quantitative ratios of diamine compound A and dicarboxylic acid compound B are selected in such a way that the molar ratio of dicarboxylic acid compound B to diamine compound A is from 0.5 to 1.5.

5: The process according to claim 1 wherein the compounds in the first reaction stage, in addition to the diamine compound A and dicarboxylic acid compound B, are an organic diol compound G, a hydroxycarboxylic acid compound H, an amino alcohol compound I, an aminocarboxylic acid compound K and/or an organic compound L which comprises at least 3 hydroxyl, primary or secondary amino and/or carboxyl groups per molecule.

6: The process according to claim 5, wherein the total amount of individual compounds G, H, I, K and L is ≦50% by weight based on the total amount of diamine compound A and dicarboxylic acid compound B.

7: The process according to claim 5, wherein the amounts of the compounds A and B and also G to L are selected such that the ratio of equivalents of the carboxyl groups and/or derivatives thereof (from the individual compounds B, H, K and L) to the sum of amino and/or hydroxyl groups and/or derivatives thereof (from the individual compounds A, G, H, I, K and L) is from 0.5 to 1.5.

8: The process according to claim 1, wherein the enzyme C is a hydrolase.

9: The process according to claim 1, wherein the enzyme C is a lipase and/or a carboxylesterase.

10: The process according to claim 1, wherein the dispersant D is a nonionic emulsifier.

11: The process according to claim 1, wherein the aqueous medium in the first reaction stage has a pH of ≧3 and ≦9.

12: The process according to claim 1, wherein the diamine compound A is 1,6-diaminohexane, 1,12-diaminododecane, 2,2-dimethyl-1,3-diaminopropane, 1,4-diaminocyclohexane, isophoronediamine, 3,3′-diaminodicyclohexylmethane, 4,4′-diaminodicyclohexylmethane, 3,3′-dimethyl-4,4′-diaminodicyclohexylmethane, m-xylylenediamine and/or p-xylylenediamine, and the dicarboxylic acid compound B is butanedioic acid, hexanedioic acid, decanedioic acid, dodecanedioic acid, terephthalic acid and/or isophthalic acid.

13: The process according to claim 1, wherein the compounds A and B and also, optionally, the compounds G to L are selected such that the polyamide obtained has a glass transition temperature of from −50 to +200° C.

14: The process according to claim 1, wherein solvent E and/or ethylenically unsaturated monomer F is used in the first reaction stage.

15: The process according to claim 1, wherein the low water solubility organic solvent E is used in an amount of from 0.1 to 40% by weight based on the total amount of water in the first reaction stage.

16: The process according to claim 1, wherein the ethylenically unsaturated monomer F but no solvent E is used in the first reaction stage.

17: The process according to claim 1, wherein the ethylenically unsaturated monomer F has a low water solubility.

18: The process according to claim 1, wherein the quantitative ratio of the sum of the total amounts of diamine compound A and dicarboxylic acid compound B to the total amount of ethylenically unsaturated monomers F is from 1:99 to 99:1.

19: The process according to claim 1, wherein the ethylenically unsaturated monomer F is a monomer mixture which comprises
from 50 to 99.9% by weight of esters of acrylic and/or methacrylic acid with alkanols having from 1 to 12 carbon atoms and/or styrene, or
from 50 to 99.9% by weight of styrene and butadiene, or
from 50 to 99.9% by weight of vinyl chloride and/or vinylidene chloride, or
from 40 to 99.9% by weight of vinyl acetate, vinyl propionate, vinyl esters of Versatic acid, vinyl esters of long-chain fatty acids and/or ethylene.

20: The process according to claim 3, wherein, on completion of the polyamide formation in the first reaction stage, the residual amounts which remain of water, dispersant D and/or ethylenically unsaturated monomer F, and also the entirety of a free-radical initiator, are added to the reaction mixture in the second reaction stage.

21: An aqueous polymer dispersion obtainable by the process according to claim 1.

22: A component in adhesives, sealants, polymer renders, papercoating slips, printing inks, cosmetic formulations and paints, for finishing leather and textiles, for fiber binding, and for modifying mineral binders or asphalt comprising the aqueous polymer dispersion according to claim 21.

23: The preparation of a polymer powder by drying the aqueous polymer dispersion according to claim 21.

24: A component in adhesives, sealants, polymer renders, papercoating slips, printing inks, cosmetic formulations, powder coatings and paints, for finishing leather and textiles, for fiber binding, and for modifying mineral binders or asphalt comprising the polymer powder prepared according to claim 23.

25: A pigment or filler in polymer formulations comprising the polymer powder prepared according to claim 23.
